(19) United States
(12) Patent Application Publication
(10) Pub. No.: US 2009/0025715 A1
(43) Pub. Date: Jan. 29, 2009

(54) Breathing Assistance Device

(76) Inventors: Travis Sugden, Stouffville (CA); Sheldon Sugden, Aurora (CA)

Correspondence Address: SIM & MCBURNEY, 330 UNIVERSITY AVENUE, 6TH FLOOR, TORONTO, ON, M5G 1R7, CA

(21) Appl. No.: 11/884,618

(22) PCT Filed: Feb. 23, 2006
(86) PCT No.: PCT/CA2006/000251

§ 371 Date: Apr. 15, 2008

Related U.S. Application Data
(60) Provisional application No. 60/655,435, filed on Feb. 24, 2005.

(51) Int. Cl. A61M 16/00

(52) U.S. Cl. 128/200.24

(57) ABSTRACT

A breathing assistance device comprises a resiliently stretchable band member dimensioned to wrap generally around a user's head; and adhesive elements removably connected to respective ends of the band member for temporarily adhering to respective cheeks of a person. During use, the band member of the device under tension exerts a tangential pull on the skin of a person's cheeks thereby to open the person's sinuses and/or nasal passages. An adhesive element for a breathing assistance device, the adhesive element comprises a base layer having a connection element for removably connecting the base layer to a platform that is connected to a respective end of a resiliently stretchable band member of the breathing assistance device; and an adhesive layer overlying at least a portion of the base layer.

### FIELD OF THE INVENTION

The disclosures herein relate generally to mechanical devices for temporarily assisting a person's breathing, and more particularly to a breathing assistance device worn about a person's head for assisting with opening a person's nasal passages.

### BACKGROUND OF THE INVENTION

Many people, typically those with deviated septa, colds, congestion, Bell's Palsy, facial muscle deterioration, excessive weight gain and the like, suffer from various breathing problems. The causes of breathing problems are varied but often include nasal congestion due to poor sinus drainage, and can lead to sleep interruption and snoring. Mechanical devices for temporarily assisting a person's breathing have been provided in the past, such as the nose-mounted dilator. The nose-mounted dilator typically includes a resilient bar that is biased to a straightened position and that has an adhesive portion on either of its ends. During use, the adhesive portion is exposed and attached to skin on either side of a person's nose, and the resilient bar is thereby caused to bend against its bias about the bridge of the nose. The resilient bar tending to straighten against its bias pulls on the skin of the person's nose, helping to open the nasal (nostril) passages. Breathing relief is thereby provided.

It has been found that the nose-mounted dilators tend to fail during use due to the adhesive breaking contact with the person's nose. This is caused by the adhesive losing its sticking power from facial oils, the manner by which the bar tends to pull the adhesive portions perpendicularly away from the person's nose, and other related factors.

U.S. Pat. No. 6,860,263 to Scoggins discloses a band nasal dilator comprising an elongated band that can wrap around a user's head. Each end of the band includes a face pad having an adhesive portion that is adapted to connect to the user's cheek. A protective tab covers the adhesive portion until the dilator is ready for use, at which time the tab can be removed to expose the adhesive portions of the face pads. In one embodiment, each face tab can include an upper and lower strip that encloses an end of the band. During use, the face tabs exert a tangential, rather than a perpendicular force on a user's cheek skin. In one embodiment, Scoggins suggests that the band and the pads can be a single integral piece of the same material.

The device disclosed by Scoggins suffers from a number of deficiencies. For example, the device is only suitable for a single use. Furthermore, Scoggins' device is not inherently adjustable in size or tension. The method by which a user employing the Scoggins device must compensate for the lack of size adjustability is to choose at which point on the user's face the pads should be attached. However, variations in user's head size will cause too much or too little tension, and/or will require placement on a portion of the user's face that despite exertion of tangential pull will not open the nasal passages. In these situations, Scoggins' device is rendered ineffective. While the user may conceivably be able to extend the length of the device a very small degree by reducing the degree to which the upper and lower layers encompass the band, doing so causes an according reduction in the amount that the face tabs grip the band; too little grip will result in a face tab breaking connection with the band, resulting in failure of the device. Furthermore, the limited size selection may only be done during the irreversible process of assembling Scoggins' band nasal dilator.

Another deficiency with Scoggins' device is that, due to its band being cylindrical, it may naturally tend to roll under tension to a location on the user's head that provides less tension to the band. Even if rolling does not occur, the band exerts directed pressure on the user's head, making the device uncomfortable to use.

It is an object of an aspect of the invention to provide a breathing assistance device that obviates or mitigates at least some of the disadvantages of the prior art.

### SUMMARY OF THE INVENTION

According to one aspect there is provided a breathing assistance device comprising:

a resiliently stretchable band member dimensioned to wrap generally around a user's head; and

adhesive elements removably connected to respective ends of the band member for temporarily adhering to respective cheeks of a person.

Each adhesive element may be supported by a respective platform, the platforms removably connected to respective ends of the band member by a respective platform retainer. The platform retainer may be a snap assembly or compatible hook-and-loop strips.

The band member may have an adjustable unstretched length.

According to another aspect, there is provided an adhesive element for a breathing assistance device, the adhesive element comprising:

a base layer having a connection element for removably connecting the base layer to a platform that is connected to a respective end of a resiliently stretchable band member of the breathing assistance device; and

an adhesive layer overlying at least a portion of the base layer.

The invention disclosed herein provides several benefits. For example, those who use the breathing assistance device are likely to experience improved sinus drainage and/or reduced snoring. Also, the adhesive elements being removably connected to the band member allows a person to repetitively re-use the band member, replacing only the adhesive elements at very small cost. In embodiments wherein the band member has an adjustable unstretched length, the person only has to adjust the band member once and for each session of use can attend to the very simple process of removably connecting another adhesive element to each end of the band member. Furthermore, the adjustability in size enables a person to tailor the device to not only their head size, but also to their particular facial topography to locate the adhesive elements in the most effective position on the person's cheeks. In embodiments where the band is wide and flat, the pressure on the person's head during use is distributed across the surface area of the band and therefore across a greater area on the user's head, providing more comfort during use.

### BRIEF DESCRIPTION OF THE DRAWINGS

Embodiments will now be described more fully with reference to the accompanying drawings, in which:

FIG. 1 is an elevational side view of a person's face and a right side of the breathing assistance device according to an embodiment;

FIG. 2 is an elevational side view of a person's face and a left side of the breathing assistance device of FIG. 1;

FIG. 3 shows side and top views of the unconnected parts of the breathing assistance device of FIGS. 1 and 2;

FIG. 4 shows side and top views of the unconnected parts of a breathing assistance device according to an alternative embodiment;

FIGS. 5a-5c show an alternative platform;

FIGS. 6a-6c show another alternative platform;

FIGS. 7a-7b show overlapping bands forming the band member, in which one band has slack thereby to regulate band member tension; and

FIGS. 8a-8b show an aromatic oil container that is attachable to the band member for providing aromas to the person during use of the breathing assistance device.

### DETAILED DESCRIPTION OF THE EMBODIMENTS

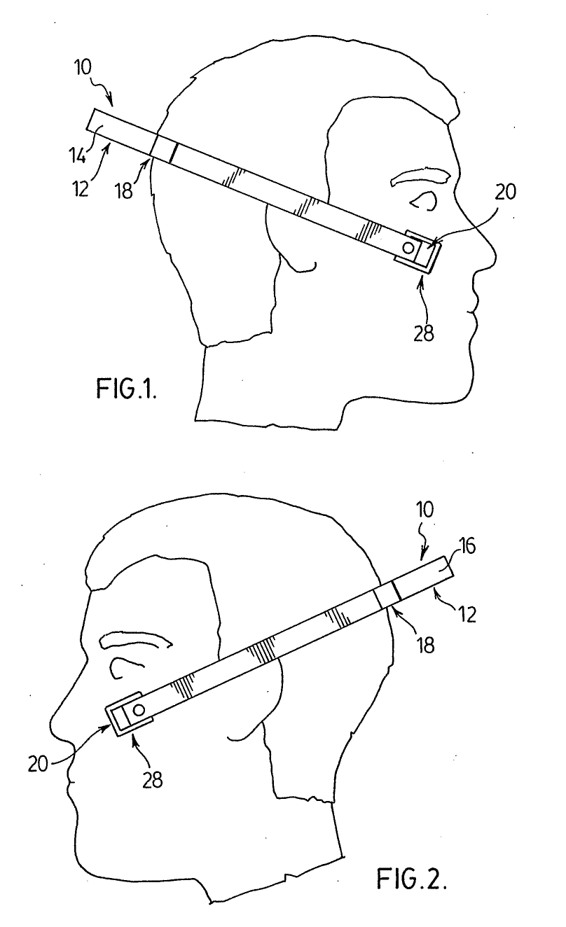

FIGS. 1 and 2 are elevational side views of a person's face and the right and left sides of a breathing assistance device 10. Breathing assistance device 10 comprises a resiliently stretchable band member 12. Band member 12 is comprised of a resiliently stretchable first band 14 and a resiliently stretchable second band 16, each being woven, wide elastic bands made of nylon and Lycra. Portions of first band 14 and second band 16 mutually overlap, and are adjustably connected with a plastic adjustable clip member 18. Adjustable clip member 18 functions to clip portions of first band 14 and second band 16 together, while enabling first band 14 and second band 16 to have adjustable amounts of overlap. The adjustable amount of overlap is provided to enable the person using breathing assistance device 10 to select a suitable unstretched length of band member 12.

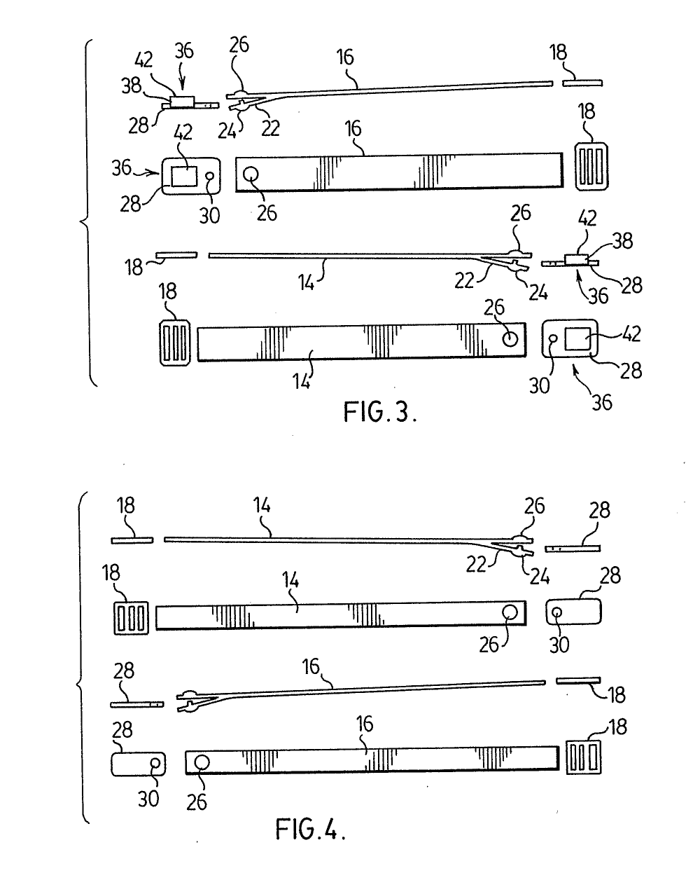

Connected to each of the two ends of band member 12, opposite the overlapping portions of first band 14 and second band 16, are respective snap assemblies 20 that function as platform retainers, as will be described. With reference to FIG. 3, which shows the unconnected parts of breathing assistance device 10 in more detail, each snap assembly 20 comprises a cloth strip 22 depending from a respective point on band member 12 a short distance from a respective end of band member 12. A male snap member 24 is attached to the end of cloth strip 22. Each snap assembly 20 also comprises a female snap member 26 attached near a respective end of band member 12 for releasably receiving male snap member 24 in snap fit, much like a button on a coat or jacket. The length of cloth strip 22 is generally such that male snap member 24 aligns with female snap member 26.

Breathing assistance device 10 also comprises two platforms 28 that are each removably connected via platform connector aperture 30 to respective snap assemblies 20 and thereby connected during use of breathing assistance device 10 to band member 12. Each platform 28 may receive an aromatic oil (not shown) for providing pleasant aromas such as strawberries, ecalyptus, lavender and the like as desired. Each platform 28 supports a respective adhesive element 36.

Each adhesive element 36 comprises a base layer 38 connected to its respective platform 28 with a connection element. Each adhesive element 36 also comprises an adhesive layer 42 for temporarily adhering to a cheek of the person. Preferably, adhesive element 36 is double-sided tape (i.e., connection element is an adhesive on one side of the tape), wherein the two sides of the double-sided tape have respective adhesive strengths in order to ensure that adhesive element 36 does not separate from its respective platform 28 during use, and is selectively removable from the person's face.

In order to use breathing assistance device 12, protective covers (not shown) are removed from each of adhesive layers 42 of adhesive elements 36 and the adhesive elements 36 are attached to respective platforms 28 with the respective connection element adhesive. Then, the respective platforms 28 are pressed against the person's cheeks adjacent their nose thereby to adhere the platforms 28 and respective adhesive elements 36 to respective cheeks adjacent the person's nose. The platforms 28 are adhered to respective cheeks of the person such that platform connection apertures 30 are generally towards the back of the person's head and platforms 28 are generally aligned with the path that band member 12 is to take around the person's head. With the platforms 28 already adhered to the person's cheeks, the male snap member 24 of one of the snap assemblies 20 is passed through a respective platform connection aperture 30 on one of the platforms 28 and then snapped into its corresponding female snap member 26.

With the one platform 28 being connected to band member 12, band member 12 is wrapped around the person's head above the ears, and the other snap assembly 20 is brought towards the remaining platform 28 that has been adhered to the person's face. In the event that band member 12 is not long enough to reach the remaining platform 28, or is too long to likely exert sufficient tension, the unstretched length of band member 12 is adjusted accordingly. The male snap member 24 of this other snap assembly 20 is then passed through the platform connection aperture 30 on the remaining platform 28 and snapped into its corresponding female snap member 26.

With the snap assemblies 20 connected to respective platforms 28, “fine-tuning” of the tension of band member 12 may be conducted so that an effective amount of upward and outward tangential pull is being applied to the skin of the person's cheeks to thereby open the nasal and/or sinus passages. The fine-tuning is done by re-adjusting the unstretched length of band member 12 as has been described.

After use, breathing assistance device 10 is removed by disconnecting platforms 28 from their respective snap assemblies 20 to separate band member 12 from platforms 28, and then disconnecting the platforms 28 and adhesive layers 42 from the person's cheeks. Alternatively, as the person prefers, the entire assembled breathing assistance device 10 may be removed simply by disconnecting the adhesive layer 42 of each adhesive element 36 from the person's cheeks. The adhesive elements 36 are then separated from platforms 28, and discarded. Platforms 28 and band member 12 are retained for another use with new adhesive elements 36. It can be seen therefore that nearly all of breathing assistance device 10 is re-usable, with the exception of adhesive elements 36. As a result, the cost for repeated use of the device is kept reasonably small.

With reference to FIG. 4, an alternative embodiment of the breathing assistance device 10 is shown, in which band member 12 is slimmer and in which a single-sided tape functions as adhesive element 36. The single-sided tape has an adhesive layer 42 and is dimensioned to stick to, and extend past the edges of, its respective platform 28. The portions of adhesive layer 42 extending past the edges of its respective platform 28 may be temporarily adhered to a user's cheeks in a similar manner as has been described. It can be seen in this embodiment that adhesive element 36's connection element would be its adhesive layer 42.

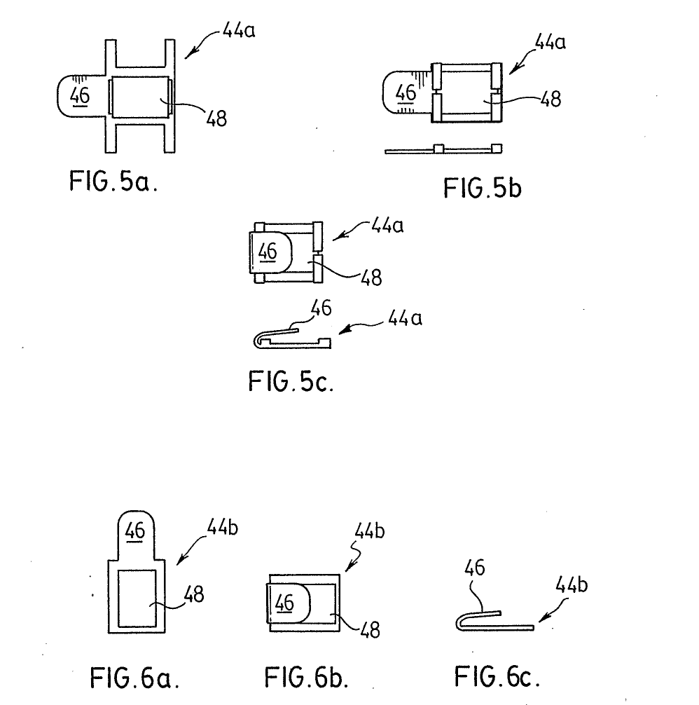

While adhesive element 36 has been described as connected to its respective platform 28 with a connection element that is an adhesive, it will be understood that other connection elements may be employed, such as mechanical connection elements. For example, with reference to FIGS. 5a-5c, an alternative platform 44 is shown made from a single piece of metal. Alternative platform 44 comprises a metal hook 46 and a hook-and-loop layer 48 retained with four (4) prongs that extend from the alternative platform 44 and are folded over hook-and-loop layer 48. Hook-and-loop layer 48 (such as provided under the trademark Velcro) functions as a platform connector in combination with another, compatible hook-and-loop layer (not shown) on a respective end of band member 12. It is to be noted that hook-and-loop layer 48 is distinct and has a different function than hook 46. Hook 46 is dimensioned to hook through a slot functioning as a connection element on an alternative adhesive element thereby to removably retain the alternative adhesive element without adhesive. As an alternative to prongs, hook-and-loop layer 48 may be connected to the metal of alternative platform 44 using a suitable glue.

FIGS. 6a-6c show another alternative platform 44 comprising a hook 46 that is made of a single piece of plastic and a hook-and-loop layer 48 glued or ultrasonically welded to the plastic.

While the adjustable overlapping connection of the first and second resiliently stretchable bands 14, 16 of band member 12 has been described as provided by adjustable clip 18, it will be understood that other means of providing the overlapping connection are possible. For example, rather than using an adjustable clip 18, overlapping portions of each of the first and second bands could be connected with regions of compatible hook-and-loop (i.e. Velcro) layers. With such a configuration, selection of the amounts of slack in the overlapping portion of one of the first and second bands 14, 16 provides a band member tension adjuster. In particular, the amount of contact between overlapping portions of first band 14 and second band 16 may be selected. More particularly, without adjusting the unstretched length of band member 12, the amount of slack on one of the bands may be manipulated so that, rather than the overlapping portions of first band 14 and second band 16 being generally parallel, the bands 14, 16 are connected such that one band has some slack. Thus, the tension can be adjusted without adjusting the unstretched length of band member 12 by, in the overlapping region, selecting the amount of slack such that tension is only due to one of the bands 14, 16 (large amount of slack), both (no slack), and somewhere in between (intermediate amount of slack).

A band member tension adjuster may also be provided by use of two (2) adjustable clips 18 instead of just one (1) such that the slack in one of the bands can be adjusted.

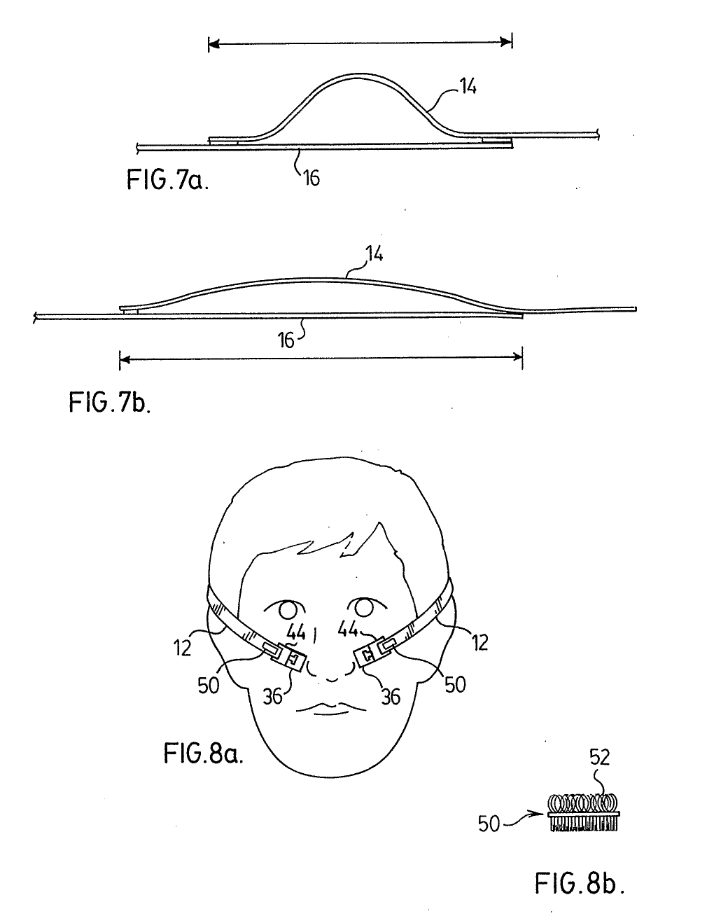

FIGS. 7a and 7b show the overlapping bands 14, 16, where band 14 has been loosely looped in order to provide slack. Upon stretching of band member 12, in overlap region band 14 having slack does not provide tension. As band member 12 is increasingly stretched, the slack is reduced until band 14 is under tension.

While snap assembly 20 has been described as comprising male and female snap members 24, 26 that are connected to either band member 12 or cloth strip 22 that depends from band member 12, it will be understood that other snap assembly configurations are possible. For example, the male and female snap members 24, 26 may be separate from band member 12 and cloth strip 22 but function in generally the same way whereby the male snap member 24 passes through cloth strip 22, platform connection aperture 30 of platform 28, and band member 12.

An alternative way to provide a pleasant aroma during use of breathing assistance device 10 is to provide one or more detachable aromatic oil containers 50 that can receive the aromatic oils. Preferably, each detachable aromatic oil container 50 is a patch of two-sided hook-and-loop material; one side being a female hook-and-loop patch, and the opposite side being a male hook-and-loop patch. The male side of each detachable aromatic oil container 50 mates with a compatible female hook-and-loop patch on a respective end of band member 12 opposite platform 28 or alternative platform 44. Aromatic oil (not shown) is applied to the female hook-and-loop patch of aromatic oil container 50, and then the male side is attached to the female hool-and-loop patch on band member 12 for enjoyment while wearing breathing assistance device 10. Advantageously, the female hook-and-loop patch side of aromatic oil container 50 functions as a matrix 52 for containing the oil while letting the aroma escape. It will be understood that other matrix materials may be employed in a similar manner.

Although embodiments have been described, those of skill in the art will appreciate that variations and modifications may be made without departing from the purpose and scope of the invention defined by the appended claims.

What is claimed is:

1. A breathing assistance device comprising:
a resiliently stretchable band member dimensioned to wrap generally around a user's head; and
adhesive elements removably connected to respective ends of the band member for temporarily adhering to respective cheeks of a person.

2. The breathing assistance device of claim 1, wherein each adhesive element is supported by a respective platform, the platforms removably connected to respective ends of the band member by a respective platform retainer.

3. The breathing assistance device of claim 2, wherein each adhesive element is a strip of double-backed tape dimensioned to be adhered to a respective platform.

4. The breathing assistance device of claim 2, wherein each platform retainer is a snap assembly connected to a respective end of the band member.

5. The breathing assistance device of claim 4, wherein each snap assembly comprises male and female snap members dimensioned to cooperate with an aperture on a respective platform.

6. The breathing assistance device of claim 5, wherein each male snap member is connected to a respective cloth strip that depends from the band member.

7. The breathing assistance device of claim 6, wherein each female snap member is connected to a respective end of the band member.

8. The breathing assistance device of claim 2, wherein each platform is made of one of plastic, Mylar, silicon and metal.

9. The breathing assistance device of claim 2, wherein each platform is removably connected to a respective end of the band member via compatible hook-and-loop layers on the platforms and the ends of the band member.

10. The breathing assistance device of claim 2, wherein each platform is formed of a single piece of metal.

11. The breathing assistance device of claim 10, wherein each platform is about 13/16 inches in length and about ⅝ inches in width.

12. The breathing assistance device of claim 2, wherein each platform is formed of a single piece of plastic.

13. The breathing assistance device of claim 12, wherein each platform is about ¾ inches in length and about ⅝ inches in width.

14. The breathing assistance device of claim 2, wherein each adhesive element comprises:
a base layer having a connection element for removably connecting the base layer to a respective platform; and
an adhesive layer overlying at least a portion of the base layer for temporarily adhering to a respective cheek of the person.

15. The breathing assistance device of claim 14, wherein the connection element is a slot and each platform comprises a hook member dimensioned to be removably received by a respective slot of a respective adhesive element.

16. The breathing assistance device of claim 14, wherein the connection element is an adhesive.

17. The breathing assistance device of claim 14, wherein each adhesive element is dimensioned to extend past edges of a respective platform, and the connection element is the adhesive layer.

18. The breathing assistance device of claim 2, wherein each platform comprises means for receiving an aromatic oil.

19. The breathing assistance device of claim 14, wherein the adhesive elements further comprise a removable protective cover overlaying the adhesive layer, wherein a user removes the removable cover layer for use.

20. The breathing assistance device of claim 1, wherein the breathing assistance device has an adjustable unstretched length.

21. The breathing assistance device of claim 20, wherein the band member comprises first and second resiliently stretchable bands having an adjustable overlapping connection.

22. The breathing assistance device of claim 21, wherein the adjustable overlapping connection is provided by an adjustable clip member.

23. The breathing assistance device of claim 21, wherein the adjustable overlapping connection is provided by two adjustable clip members forming a band member tension adjuster.

24. The breathing assistance device of claim 23, wherein the band member tension adjuster provides selectable amounts of slack in an overlapping portion of one of the first and second bands.

25. The breathing assistance device of claim 21, wherein the adjustable overlapping connection is provided by compatible hook-and-loop layers on the bands forming a band member tension adjuster.

26. The breathing assistance device of claim 25, wherein the band member tension adjuster provides selectable amounts of slack in an overlapping portion of one of the first and second bands.

27. The breathing assistance device of claim 1, further comprising:
at least one aromatic oil container removably connected to a respective end of the band member.

28. The breathing assistance device of claim 27, wherein the at least one aromatic oil container comprises:
a first hook-and-loop patch compatible with a second hook-and-loop patch on a respective end of the band member opposite the adhesive element;
a matrix layer associated with the first hook-and-loop patch for receiving and containing an aromatic oil.

29. The breathing assistance device of claim 28, wherein the matrix layer is a female hook-and-loop patch.

30. An adhesive element for a breathing assistance device, the adhesive element comprising:
a base layer having a connection element for removably connecting the base layer to a platform that is connected to a respective end of a resiliently stretchable band member of the breathing assistance device; and
an adhesive layer overlying at least a portion of the base layer.

31. The adhesive element of claim 30, wherein the connection element comprises a slot dimensioned to receive a hook member on the platform.

32. The adhesive element of claim 30, wherein the connection element comprises an adhesive.
